(19) United States
(12) Patent Application Publication
(10) Pub. No.: US 2015/0315566 A1
(43) Pub. Date: Nov. 5, 2015

(54) METHOD FOR GENERATING HIGH AFFINITY, BIVALENT BINDING AGENTS

(71) Applicant: The Board of Trustees of the University of Illinois, Urbana, IL (US)

(72) Inventors: Brian K. Kay, Chicago, IL (US); Kevin T. Gorman, Chicago, IL (US); Renhua Huang, Rockville, MD (US)

(21) Appl. No.: 14/698,158

(22) Filed: Apr. 28, 2015

Related U.S. Application Data
(60) Provisional application No. 61/986,192, filed on Apr. 30, 2014.

(51) Int. Cl. C12N 15/10; C12N 15/86

(52) U.S. Cl. CPC C12N 15/1037; C12N 15/86; C12N 15/1058

(57) ABSTRACT

A combined Kunkel mutagenesis and phage-display method for producing bivalent binding agents is provided.

### INTRODUCTION

This application claims the benefit of priority of U.S. Provisional Application Nos. 61/986,192, filed Apr. 30, 2014, the content of which is incorporated herein by reference in its entirety.

This invention was made with government support under contract number 1 U54 DK093444 awarded by the National Institutes of Health. The government has certain rights in the invention.

### BACKGROUND

In the field of antibody engineering, it is often desirable to generate bivalent affinity reagents because of their useful properties including impressive binding affinity and use in sandwich ELISA (Silverman, et al. (2005) Nat. Biotechnol. 23:1556-61; Lee, et al. (2004) J. Immunol. Meth. 284:119-132; Drow, et al. (1979) J. Clin. Microbiol. 10:9). The traditional approach for isolating a pair of binders to non-overlapping epitopes (on the target protein) has been laborious. One must perform affinity selection, additional mutagenesis, further affinity selection, and finally epitope binning to find binding pairs that interact with different epitopes (Abdiche, et al. (2009) Anal. Biochem. 386:172-80). In addition to being time consuming, this approach is also not amenable to high-throughput strategies and is therefore not cost effective.

Several techniques are available for preparing antibody variants via site-directed mutagenesis. Cassette mutagenesis (Wells & Estell (1988) Trends Biochem. Sci. 13:291-297), which requires restriction enzyme digestion and ligation to incorporate mutagenic sequences, has been supplanted by the QUIKCHANGE method (Vandeyar, et al. (1988) Gene 65:129-133; Sugimoto, et al. (1989) Anal. Biochem. 179:309-11). In QUIKCHANGE, a pair of complementary oligonucleotides, containing the desired mutation(s), is used to amplify the entire plasmid with a high-fidelity polymerase, followed by DpnI digestion to remove the parental strand. A third widely used technique is Kunkel mutagenesis (Kunkel (1985) Proc. Natl. Acad. Sci. USA 82:488-92; Kunkel, et al. (1987) Methods Enzymol. 154:367-382; Scholle, et al. (2005) Comb. Chem. High Throughput Screen. 8:545-551; Tonikian, et al. (2007) Nature Protocols 2:1368-1386; Wojcik, et al. (2010) Nature Struct. Mol. Biol. 17:519-527) and derivatives thereof (See US 2013/0045507), where uracil-inserted, circular, single-stranded DNA (ssDNA) is used as a template to synthesize double-stranded DNA (dsDNA) in vitro with a short oligonucleotide primer that introduces a mutation. After dsDNA is introduced into bacteria, recombinant clones predominate due to cleavage of the uracilated strand in vivo. Kunkel mutagenesis has been useful in phage-display experiments that are based on M13 or related phage, as the viral particles contain a circular, single-stranded genome (Scholle, et al. (2005) Comb. Chem. High Throughput Screen. 8:545-551; Fellouse, et al. (2007) J. Mol. Biol. 373:924-9401; Huang et al. (2012) N. Biotechnol. 29(5):526-33; U.S. Pat. No. 8,685,893).

As the number of the theoretical permutations in a protein engineering experiment can be astronomical, it is desirable to construct phage-displayed libraries that include a vast number of mutants, as it has been observed that the size of a phage library is closely correlated with the affinity of the isolated mutants (Ling (2003) Comb. Chem. High Throughput Screen. 6:421-432). While the size of the library is a limiting factor in isolating desired clones, the quality of the phage library (i.e., the percentage of the phage particles displaying the recombinant polypeptides out of the total phage pool), also significantly influences the efficiency and the outcome of affinity selections. For example, some studies have found that non-recombinant clones, or target-unrelated clones, can overwhelm the target-binding clones in the library due to the advantages associated with steps of phage propagation or affinity selection (Menendez & Scott (2005) Anal. Biochem. 336:145-157; Brammer, et al. (2008) Anal. Biochem. 373:88-98).

Even with improvements in the size and quality of a phage-displayed library, affinity maturation experiments are usually necessary to fine-tune binders for improved specificity (Huang et al. (2012) N. Biotechnol. 29(5):526-33; Fagete, et al. (2009) MAbs. 1:288-296), affinity (Huang et al. (2012) N. Biotechnol. 29(5):526-33; Yang, et al. (1995) J. Mol. Biol. 254:392-403; Groves, et al. (2006) J. Immunol. Methods 313:129-139), or both (Huang et al. (2012) N. Biotechnol. 29(5):526-33). One simple method is to generate secondary (i.e., mutant) libraries through an error-prone polymerase chain reaction (PCR) (Gram, et al. (1992) Proc. Natl. Acad. Sci. USA 89:3576-3580; Daugherty, et al. (2000) Proc. Natl. Acad. Sci. USA 97:2029-2034), and repeat the affinity selections under more stringent conditions (i.e., less target, longer wash times, more washes). Nevertheless, generating each secondary library can be time-consuming, and unless large, may be inadequate for isolating mutants with dramatically improved properties.

Alternative approaches for generating bivalent molecules having a high affinity and specificity for a given target have been described. For example, US 2011/0143963 describes affinity reagents, termed “modular molecular affinity clamps,” that have a clamp-like or clamshell architecture and are composed of two discrete modules, each of which bind the same target peptide motif. Further, Jäger, et al. ((2013) BMC Biotechnol. 13:52) describe scFv-Fc antibody fusion proteins, which can be transiently expressed and screened in a high-throughput recombinant manner.

### SUMMARY OF THE INVENTION

This invention is a method for generating a high affinity, bivalent binding agent to a target molecule. The method involves the steps of (a) obtaining nucleic acids encoding a population of binding agents that bind to a target molecule; (b) amplifying the nucleic acids of (a) to generate a pool of megaprimers; (c) annealing the pool of megaprimers of (b) to a single-stranded, uracilated phage-display vector comprising a first binding agent coding region and second binding agent coding region each capable of hybridizing to the pool of megaprimers, wherein the first binding agent coding region and second binding agent coding region are in tandem and linked via a nucleic acid encoding a flexible linker; (d) primer extending the annealed product of (c) to generate a phage-display library bivalent phage clones; and (e) screening the phage-display library to identify a bivalent phage clone that binds to the target molecule. In some embodiments, the population of binding agents includes a library of antibody fragments of antibodies, single-domain antibodies, Forkhead-Associated domains, monobodies, minibodies, single-chain variable fragments, AFFIBODY molecules, affilins, anticalins, designed ankyrin repeat proteins, nanofitins, linear peptides or a combination thereof. A phage-display vector and kit are also provided. In some embodiments, the first binding agent coding region and second binding agent coding region of the vector include at least one stop codon. In other embodiments, the first binding agent coding region and second binding agent coding region of the vector are the same or different.

### BRIEF DESCRIPTION OF THE DRAWINGS

FIG. 1 depicts the inventive method of megaprimer shuffling for tandem affinity reagents (MegaSTAR). Megaprimers are created by PCR amplification of the coding region for a pool of pre-selected clones (Steps A and B). The megaprimers are then annealed to a single-stranded, uracilated tandem DNA template containing two coding regions (Step C). The recombinant strand is then filled in via DNA polymerase and transformed into E. coli. The bacterium then degrades the uracilated parent strand, leaving the recombinant strand that now allows the phage to display tandem, linked binders (Steps D and E). When performed with a population of clones, this creates a new “bivalent library” of use in subsequent affinity selection to isolate bivalent reagents with the strongest affinity for the target.

FIG. 2 depicts the general structure of a phage-display vector for use in generating a FN3-based bivalent binding agent.

FIG. 3 shows the nucleotide (SEQ ID NO:16) and deduced amino acid sequence (SEQ ID NOs:17-20) of a portion of a phage-display vector encoding tandem FN3 proteins. Stop codons (TAA and TGA) and StuI restriction sites introduced into each BC and FG loop region of the FN3 coding sequences are indicated. Myc and FLAG tags and linker sequences are also indicated.

### DETAILED DESCRIPTION OF THE INVENTION

It has now been shown that Kunkel mutagenesis and phage-display can be combined to generate high affinity bivalent binding agents. The resulting binding agents are unique in that they bind to two distinct epitopes on the same target protein. The present method allows for the rapid and efficient generation of these binding agents in a high throughput manner to any target of interest. This invention converts the output of an affinity selection to a bivalent display format via a technology referred to herein as Megaprimer Shuffling for Tandem Affinity Reagents (MegaSTAR). This method relies on synthesizing a “bivalent library” by first generating a pool of megaprimers from a selection output, and annealing them randomly to a bivalent vector. This new “bivalent library” is then used for further affinity selection to identify tandem reagents with the highest affinity. This allows for the examination of many different combinations of binding agents simultaneously, thereby eliminating the need for pair-wise clonal analysis. Ultimately, this method greatly decreases the time and cost of creating bivalent binding agents of use, e.g., in diagnostic, therapeutic and laboratory applications.

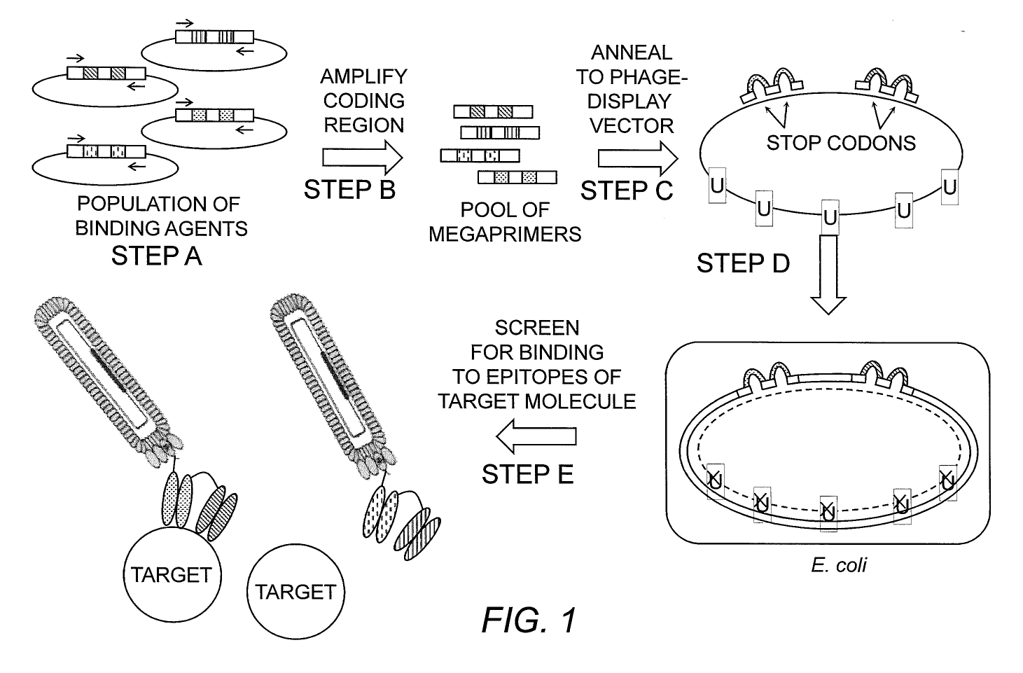

Accordingly, the present invention is a method for generating a high affinity, bivalent binding agent to a target molecule by (a) obtaining nucleic acids encoding a population of binding agents that bind to a target molecule; (b) amplifying the nucleic acids of (a) to generate a pool of megaprimers; (c) annealing the pool of megaprimers of (b) to a single-stranded, uracilated phage-display vector harboring a first binding agent coding region and second binding agent coding region each capable of hybridizing to the pool of megaprimers, wherein the first binding agent coding region and second binding agent coding region are in tandem and linked via a nucleic acid encoding a flexible linker; (d) primer extending the annealed nucleic acids of (c) to generate a phage-display library of bivalent phage clones (via Kunkel mutagenesis); and (e) screening the phage-display library to identify a bivalent phage clone that binds to the target molecule thereby generating a high affinity bivalent binding agent. For simplicity, the method of the invention is described for use in generating a high affinity bivalent binding agent to a single target molecule of interest. See the illustrative example depicted in FIG. 1. However, it is also contemplated that the method of the invention can be used to generate a high affinity bivalent binding agent that binds to two distinct target molecules of interest.

For the purposes of the present invention, a “binding agent” refers to a protein that has a high affinity for, and specifically binds to, a target molecule, e.g., an antigen. A “bivalent binding agent” refers to a binding agent that binds to two different epitopes. In some embodiments, the epitopes are present on the same antigen or target molecule of interest. In other embodiments, the epitopes are present on two different antigens or target molecules of interest.

As used herein, the term “affinity” refers to the non-random interaction of two molecules. Affinity, or the strength of the interaction, can be expressed quantitatively as a dissociation constant (K_{D}). Binding affinity can be determined using standard techniques. In particular embodiments, the binding agents of this invention have a high affinity for a target molecule, with K_{D}s in the range of low μM (e.g., 1-10 μM) to nM, or more preferably in the range of nM to pM.

Binding agents in accordance with this invention are artificial proteins that are composed of fragments of antibodies (e.g., Fab and Fd fragments), single-domain antibodies, Forkhead-Associated (FHA) domains, monobodies, minibodies, single-chain variable fragments (scFv), AFFIBODY molecules, affilins, anticalins, DARPins (i.e., designed ankyrin repeat proteins), and nanofitins (also known as affitins). Other binding agents that can be generated using this method include receptors, enzymes, peptides and protein ligands. In certain embodiments, the binding agent is a single chain molecule and/or monomeric. Desirably, the binding agent of the invention is in the range of 5 to 800 amino acid residues in length, or more desirably 60 to 600 amino acid residues in length. Moreover, the binding agent is preferably thermal stable, lacks cysteine residues, can be expressed via a recombinant expression system (e.g., E. coli), has a known three-dimensional structure, has uniform biochemical properties among variants, and/or can bind to one or more target molecules.

Single-domain antibodies or nanobodies are antibody fragments composed of a single monomeric variable antibody domain (Harmsen & De Haard (2007) Appl. Microbiol. Biotechnol. 77:13-22). Like a whole antibody, it is able to bind selectively to a specific antigen. Single-domain antibodies are typically ˜110 amino acid residues long and can be derived from heavy-chain antibodies found in camelids (i.e., V_{H}H fragments) or cartilaginous fish (i.e., V_{NAR}). An alternative approach is to split the dimeric variable domains from common IgG from humans or mice into monomers (Holt, et al. (2003) Trends Biotechnol. 21:484-490). As with antibodies, the CDRs of nanobodies can be modified to alter the specificity of the nanobodies.

The Forkhead-Associated domain is a phosphopeptide recognition domain found in many regulatory proteins (Hofmann & Buchner (1995) Trends Biochem. Sci. 20:347-9). FHA domains are approximately 65-100 amino acid residues and display specificity for phosphothreonine-containing epitopes, but can also recognize phosphotyrosine with relatively high affinity. The FHA domain forms an 11-stranded β-sandwich that has a short α-helix inserted between β strands 2 and 3 and an α-helical region at the extreme C-terminus. The peptide binding site is created by the loop regions between β3/4, β4/5, and β6/7 (Durocher, et al. (2000) Mol. Cell 6:1169-1182), which can modified to alter specificity and affinity.

Monobodies, also known as Adnectins, are 94 amino acid proteins, which are based upon the structure of human fibronectin, in particular the tenth extracellular type III domain of fibronectin. This domain, referred to as the FN3 scaffold, has a structure similar to antibody variable domains, with two β-sheets, one constituted by β-strands A, B and E, and the other by β-strands C, D, F and G (Koide & Koide (2007) Methods Mol. Biol. 352:95-109). The specificity of monobodies can be tailored by modifying the loops BC (between the second and third beta sheets) and FG (between the sixth and seventh sheets)(Koide, et al. (1998) J. Mol. Biol. 284:1141-51). An exemplary FN3 monobody scaffold has the amino acid sequence:

(SEQ ID NO: 1)
VSDVPRDLEV VAATPTSLLI SWDAPAVTVR YYRITYGETG
GNSPVQEFTV PGSKSTATIS GLKPGVDYTI TVYAVTGRGD
SPASSKPISI NYRT.

See U.S. Pat. No. 6,818,418. Another exemplary FN3 monobody scaffold includes the sequence:

(SEQ ID NO: 2)
MAVSDVPRKL EVVAATPTSL LISWDAPCRK CLYYRITYGE
TGGNSPVQEF TVPGSKSTAT ISGLKPGVDY TITVYAVTRL
EFISKPIISI NYRI.

A minibody scaffold, which is related to the immunoglobulin fold, is a protein generated by deleting three beta strands from a heavy chain variable domain of a monoclonal antibody (Tramontano, et al. (1994) J. Mol. Recognit. 7:9). This protein includes 61 residues and can be used to present two hypervariable loops. In some embodiments, a minibody is a homodimer, wherein each monomer is a single-chain variable fragment (scFv) linked to a human IgG1 CH3 domain by a linker, such as a hinge sequence.

Single-chain variable fragments (scFv) are fusion proteins composed of the variable regions of the heavy (V_{H}) and light (V_{L}) chains of immunoglobulins, which are connected by a short peptide of ten to about 25 amino acid residues. The peptide is usually rich in glycine for flexibility, as well as serine or threonine for solubility, and can either connect the N-terminus of the V_{H} with the C-terminus of the V_{L}, or vice versa. As with antibodies, the CDRs of scFv molecules can be modified to alter the specificity of the scFv.

Affibody molecules are small proteins (e.g., 58 amino acid residues) with a three-helix bundle domain, originally based upon the Z domain of staphylococcal protein A (Ståhl & Nygren (1997) Pathol. Biol. (Paris) 45:66-76; Nilsson, et al. (1987) Prot. Eng. 1:107-133; and U.S. Pat. No. 5,143,844). Based on the Z protein as a basic structure or scaffold, mutagenesis of surface-exposed amino acids can be carried out to create variants with an altered binding affinity. See, U.S. Pat. No. 6,534,628; Nord, et al. (1995) Prot. Eng. 8:601-608; Nord, et al. (1997) Nat. Biotech. 15:772-777.

Affilins are proteins that are structurally derived from human gamma-B crystallin or ubiquitin. The binding region of affilins is located in a beta sheet (Ebersbach, et al. (2007) J. Mol. Biol. 372:172-185; Vijay-Kumar, et al. (1987) J. Mol. Biol. 194:531-44), such that modification of near-surface amino acid residues of these proteins alters specificity. In particular, the near surface amino acids 2, 4, 6, 15, 17, 19, 36 and 38 of gamma crystalline are typically modified (see, WO 01/04144), whereas residues 2, 4, 6, 62, 63, 64, 65 and 66 of ubiquitin are typically modified (see, WO 2006/040129).

Anticalins are artificial proteins derived from lipocalins, which can bind to either proteins or small molecules (Weiss & Lowman (2000) Chem. Biol. 7:547-554). Lipocalins of use in this invention include, but are not limited to the bilin-binding protein (BBP) from Pieris brassicae (Beste, et al. (1999) Proc. Natl. Acad. Sci. USA 96:1898-1903; Schmidt & Skerra (1994) Eur. J. Biochem. 219:855-863; Schlehuber, et al. (2000) J. Mol. Biol. 297:1105-1120) and bovine retinol-binding protein (RBP) (Berni, et al. (1990) Eur. J. Biochem. 192:507-513). Anticalins have a barrel structure formed by eight antiparallel β-strands pairwise connected by loops. Sixteen 16 amino acid residues, distributed across the four loops, form the binding site, which can be mutagenized to modify affinity and selectivity (Skerra (2008) FEBS J. 275:2677-83).

DARPins derived from ankyrin proteins are composed of at least three, usually four or five repeat motifs, and have a molecular mass of about 14 to 18 kDa. Using a combination of sequence and structure consensus analyses, a amino acid residue ankyrin repeat module with seven randomized positions has been developed as a binding agent (Binz, et al. (2003) J. Mol. Biol. 332:489-503).

Nanofitins are 66 amino acid residue proteins derived from the DNA binding protein Sac7d of Sulfolobus acidocaldarius (EP 2469278). The binding area of nanofitins is located on the surface and is composed of 14 residues (i.e., residues 7-9, 21, 22, 24, 26, 29, 31, 33, 40, 42, 44, and 46), which can be modified to alter specificity (Mouratou, et al. (2007) Proc. Natl. Acad. Sci. USA 104:17983-8).

In accordance with the method of the invention, nucleic acids encoding a population of binding agents that bind to a target molecule are obtained (See FIG. 1, Step A). The nucleic acids of said population can encode a single type of binding agent, e.g., antibody fragments, single-domain antibodies, FHA domains, monobodies, minibodies, scFv, AFFIBODY molecules, affilins, anticalins, DARPins or nanofitins. Alternatively, the population of binding agents can encode a mixture of said binding agents that bind to a target molecule of interest. By way of illustration, the population of binding agents can include a mixture of monobodies and DARPins; monobodies and AFFIBODY molecules; single-domain antibodies and DARPins; monobodies, affilins and DARPins; etc. In this respect, a portion of the bivalent binding agent will be one type of binding agent molecule and the other portion of the bivalent binding agent will be another type of binding agent molecule.

Desirably, the population of binding agents that bind to a target molecule are obtained from a primary selection or primary library screen for binding agents that bind to the target molecule of interest. For example, a library containing a diverse population of binding agents can be screened for binding to a target molecule of interest and binding agents that exhibit varying degrees of affinity for the target molecule can be pooled to create a population of binding agents of use in the method of the invention. Libraries can include, e.g., yeast, bacteria, bacteriophage or phagemid, virus, cell, ribosome, or a combination of such in vitro display systems. An exemplary cell-free display system is described in WO 01/05808. A ribosome display library is described by Groves, et al. ((2006) J. Immunol. Meth. 313:129-139). Phage display technology is well-known in the art (see, for example, WO 91/17271 or WO 92/001047). Phage used in such methods are typically filamentous phage including fd and M13 binding domains expressed from phage with the binding agent protein recombinantly fused to either the phage gene III or gene VIII protein.

Once the population of binding agents that bind to a target molecule has been obtained, the nucleic acids encoding the binding agents are amplified to generate a pool of megaprimers (See FIG. 1, Step B). A megaprimer for the purposes of the present invention is intended to refer to an oligonucleotide that is in the range of, e.g., 100 to 1000 or more bases in length, and is capable of serving as a primer for enzymatic extension of nucleic acid molecules (e.g., the megaprimer is phosphorylated). Amplification of the nucleic acids encoding the binding agents can be carried out e.g., by conventional reverse-transcription (if the nucleic acids encoding the population of binding agents is RNA) and/or PCR amplification using primers, which flank the nucleic acids encoding the binding agent (e.g., vector sequences) or primers that hybridize to the 3′ and 5′ ends of the nucleic acids encoding the binding agent. Such primers can be readily designed by the skilled artisan based upon the known nucleic acid sequences encoding antibody fragments, single-domain antibodies, FHA domains, monobodies, minibodies, scFv, AFFIBODY molecules, affilins, anticalins, DARPins and nanofitins. For example, PCR amplification of the sequence encoding the 10^{th} subunit of human fibronectin type III repeat (FN3) has been described by Karatan, et al. ((2004) Chem. Biol. 11:835-44). Likewise, primer combinations for amplifying a majority of the known human antibody sequences is described by Yuan, et al. ((2013) Neural Regen. Res. 8:3107-3115).

Given that the present method is of use in generating a high affinity, bivalent binding agent with altered affinity and/or specificity for a target molecule, and/or altered solubility, protein folding, thermal stability etc., the step of amplifying the nucleic acids encoding the binding agents can be carried out via, e.g., error-prone PCR (Zaccolo, et al. (1996) J. Mol. Biol. 255:589-603) or mutagenic PCR (Cadwell & Joyce (1994) PCR Methods Appl. 3:S136-S140) to add more diversity through random mutation.

In the subsequent step of the method of the invention, the pool of megaprimers are used in Kunkel mutagenesis (Kunkel, et al. (1991) Methods Enzymol. 204:125-139; Huang, et al. (2012) Methods 58:10-17) to prime DNA synthesis with an phage-display vector as a template (See FIG. 1, Step C). To facilitate subsequent screening, certain embodiments include removal of the template strand. This can be achieved by restriction enzymatic digestion and/or using an uracilated template strand, as exemplified herein. For example, the template phage-display vector can be replicated in the presence of uracil in an ung-/dut- E. coli strain, such as BW313 and CJ236, which are deficient in the enzyme dUTP pyrophosphatase (dut-) resulting in an increased incorporation of uracil in place of thymine in the DNA. Uracilated template DNA may also be prepared enzymatically using for example T7 DNA polymerase together with dNTP's and dUTP. Uracilated template DNA can then be removed by treatment with uracil-N-glycosylase (UDG), which hydrolyzes uracils in the heteroduplex.

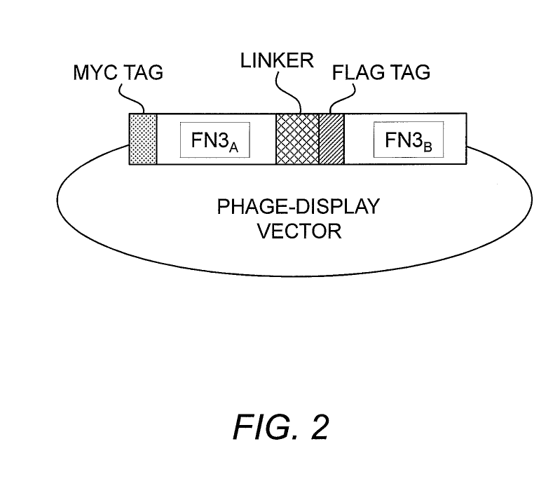

To serve as a template for generating a bivalent binding agent, the nucleic acids of the phage-display vector include harbor a first binding agent coding region and a second binding agent coding region, each of which is capable of hybridizing to a member of the pool of megaprimers (see FIG. 2). In this respect, the first and second binding agent coding regions of the phage-display vector have one or more contiguous nucleic acid sequences that are complementary to sequences present in the megaprimers. Such complementary sequences can, e.g., be non-random sequences that are outside of the variable region sequences of the binding agents. Typically, there are about 15 to about 30 complementary nucleotides in the 5′ end and about 15 to about 30 complementary nucleotides in the 3′ end of the megaprimer relative to the first and second binding agent coding regions of the phage-display vector. However, shorter complementary segments may also be used. During this step, each of the megaprimers obtained in the previous step has an equal chance to anneal to the first and second binding agent coding regions within the phage-display vector, thus allowing many different combinations of binding agents from the population of binding agents to now be linked together as bivalent molecules.

To ensure that the first and second binding agent coding regions do not interfere with subsequent screening for high affinity binding agents, the first and second binding agent coding regions further encode one or more stop codons (see FIG. 2). In certain embodiments, the stop codons are located within sequences encoding the variable region of the binding agent. In other embodiments, sequences encoding the variable region of the binding agent have restriction endonuclease recognition sites. As a result of annealing the megaprimers to the uracilated phage-display vector, the first binding agent coding region forms a heteroduplex with a first megaprimer and the second binding agent coding region forms a heteroduplex with a second megaprimer (See FIG. 1, Step C).

In some embodiments, the bivalent binding agent is composed of a single type of binding agent, e.g., antibody fragments, single-domain antibodies, FHA domains, monobodies, minibodies, scFv, AFFIBODY molecules, affilins, anticalins, DARPins or nanofitins. Therefore, in accordance with this embodiment, the first and second binding agent coding regions of the phage-display vector have substantially the same sequence.

In other embodiments, the bivalent binding agent is composed of a mixture of binding agents that bind to a target molecule of interest. Therefore, the first and second binding agent coding regions of the phage-display vector are different. By way of illustration, the first binding agent coding region can encode a monobody, whereas the second binding agent coding region encodes for a DARPin.

In further embodiments, the phage-display vector encodes a tag located upstream or downstream of one or both of the first and second binding agent coding regions. Such tags may be useful in purification, detection and/or screening and include, but not limited to, a FLAG-tag, polyhistidine-tag, a gD-tag, a c-myc tag, green fluorescence protein tag, a GST-tag or β-galactosidase tag. In certain embodiments, it is contemplated that a tag is located upstream of both the first and second binding agent coding regions, and said tags are different from one another.

To generate a bivalent binding agent, the first and second binding agent coding regions are tandemly arranged in the phage-display vector and are linked via a nucleic acid molecule encoding a flexible linker. Linkers of use in this invention can be between 5 and 50 amino acid residues in length and can be selected from any suitable linker known in the art. See, e.g., LeGall, et al. (2004) Prot. Eng. Design Select. 17:357-366. Exemplary linkers include, but are not limited to, GGGGSGGGGSGGGGS (i.e., (G₄S)₃)(SEQ ID NO:3), GGGGSGGGGSGGGGSGGGGSGGGGS (i.e., (G₄S)₅)(SEQ ID NO:4), SAKTTPKLGG (SEQ ID NO:5), RADAAPTVS (SEQ ID NO:6), RADAAAAGGPGS (SEQ ID NO:7), and RADAAAA(G₄S)₄ (SEQ ID NO:8). In certain embodiments, the flexible linker of the bivalent binding agent contains a (GGGGS)ₙ motif (SEQ ID NO:9) (Chen, et al. (2013) Adv. Drug Deliv. Rev. 65:1357-69).

A phage-display vector of the present invention is a vector containing phage-derived polynucleotide sequences capable of expressing, or conditionally expressing, a heterologous polypeptide, for example, as a fusion protein with a phage protein (e.g., a phage surface protein). In some embodiments, a phage-display vector of the present invention is a vector derived from a filamentous phage (e.g., phage f1, fd, and M13) or a bacteriophage (e.g., T7 bacteriophage or a lambdoid phage. Filamentous phage and bacteriophage are described by, e.g., Santini ((1998) J. Mol. Biol. 282:125-135), Rosenberg, et al. ((1996) Innovations 6:1-6), and Houshmand, et al. ((1999) Anal. Biochem. 268:363-370).

In general, a phage-display vector of the invention can include the following elements: a promoter suited for constitutive or inducible expression (e.g., lac promoter); a ribosome binding site and signal sequence preceding the sequence encoding a displayed peptide; and one or more restriction sites; optionally, a tag sequence such as a stretch of 5-6 histidines or an epitope recognized by an antibody; a second tag sequence; a suppressible codon (e.g., a termination codon); and a sequence encoding a phage surface protein positioned in-frame to form a fusion with the bivalent binding agent to be displayed. In general, a phage-display vector of the invention contains a promoter and/or regulatory region operably linked to a polynucleotide sequence encoding the bivalent binding agent and a sequence encoding a phage surface protein.

The term “operably linked” refers to a functional linkage between nucleic acid sequences such that the linked promoter and/or regulatory region functionally controls expression of the coding sequence. It also refers to the linkage between coding sequences such that they may be controlled by the same promoter and/or regulatory region. Such linkage between coding sequences may also be referred to as being linked in-frame or in the same coding frame such that a fusion protein comprising the amino acids encoded by the coding sequences may be expressed.

In other embodiments of the invention, the ability of the phage-display vector to express a fusion protein is regulated in part by use of a regulated promoter or other regulatory region (e.g., an inducible promoter such that in the absence of induction, expression is low or undetectable). Non-limiting examples of inducible promoters include the lac promoter, the lac UV5 promoter, the arabinose promoter, and the tet promoter. In some embodiments, an inducible promoter can be further restricted by incorporating repressors (e.g., lacl) or terminators (e.g., a tHP terminator). For example, repressor lacl can be used together with the Lac promoter.

As used herein, the term “phage surface protein” refers to any protein normally found at the surface of a filamentous phage (e.g., phage f1, fd, and M13) or a bacteriophage (e.g., λ, T4 and T7) that can be adapted to be expressed as a fusion protein with a heterologous polypeptide and still be assembled into a phage particle such that the polypeptide is displayed on the surface of the phage. Suitable surface proteins derived from filamentous phages include, but are not limited to, minor coat proteins from filamentous phages, such as gene III proteins and gene VIII proteins; major coat proteins from filamentous phages such as, gene VI proteins, gene VII proteins, gene IX proteins; gene 10 proteins from T7; and capsid D protein (gpD) of bacteriophage A. In some embodiments, a suitable phage surface protein is a domain, a truncated version, a fragment, or a functional variant of a naturally occurring surface protein. For example, a suitable phage surface protein can be a domain of the gene III protein, e.g., the anchor domain or “stump.” Additional exemplary phage surface proteins are described in WO 00/71694. As appreciated by the skilled artisan, the choice of a phage surface protein is to be made in combination with a consideration of the phage-display vector and the cell to be used for propagation thereof.

Any peptide sequences capable of driving or directing secretion of expressed protein or polypeptide can be used as signal sequence for the phage-display vector of the invention. Exemplary leader sequences include, but not limited to, a PelB leader sequence and an OmpA leader sequence.

General methods for constructing phage-display vectors, phage-display libraries and the method of use are described, for example, in U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard, et al. (1999) J. Biol. Chem. 274:18218-30; Hoogenboom, et al. (1998) Immunotechnology 4:1-20; Hoogenboom, et al. (2000) Immunol. Today 2:371-8; Fuchs, et al. (1991) Bio/Technology 9:1370-1372; Huse, et al. (1989) Science 246:1275-1281; Griffiths, et al. (1993) EMBO J. 12:725-734; Hawkins, et al. (1992) J. Mol. Biol. 226:889-896; Clackson, et al. (1991) Nature 352:624-628; Gram, et al. (1992) PNAS 89:3576-3580; Garrard, et al. (1991) Bio/Technology 9:1373-1377; Rebar, et al. (1996) Methods Enzymol. 267:129-49; Hoogenboom, et al. (1991) Nucl. Acid Res. 19:4133-4137; and Barbas, et al. (1991) PNAS 88:7978-7982.

Once the reverse-transcribed/PCR-amplified megaprimers are annealed to the uracilated phage-display vector the megaprimer is extended in the presence of a DNA polymerase (e.g., T7 or T4 polymerase) and ligated with a DNA ligase (e.g., T4 ligase) to generate a heteroduplex phage-display library, i.e., covalently closed circular DNA (cccDNA), in which one stand is the template strand and the other, in vitro synthesized strand, encodes a bivalent binding agent of interest (see FIG. 1, Step D). Upon transformation into an appropriate host cell (e.g., E. coli), the uracilated parent strand (containing stop codons and restriction sites within the coding region) is degraded by the host cell leaving the recombinant strand coding for a pair of binding agents that are now linked.

Once prepared, the phage-display library is subsequently screened to identify bivalent phage clones of the library that bind to the target molecule of interest (see FIG. 1, Step E). The phage-display library screening step is carried out by inducing the phage to display the bivalent binding agents on the surface of the phage clones and identifying bivalent binding agents that bind to the target molecule. Any suitable method that detects interactions between molecules can be used to identify bivalent binding agents of interest including, e.g., ELISA, co-immunoprecipitation, bimolecular fluorescence complementation, affinity electrophoresis, pull-down assays, label transfer, and the like. However, in certain embodiments, the screen is carried out using a tagged target molecule. For example, a biotin-tagged target molecule can be contacted with the library of phage-displayed bivalent binding agents, and bivalent binding agent-target molecule complexes are captured using streptavidin-coated magnetic beads. Advantageously, any unbound phage-displayed bivalent binding agents can be removed by washing the magnetic beads.

Target molecules that can be used in accordance with this invention include proteins, glycoproteins, phosphoproteins, other post-translationally modified proteins, protein complexes, nucleic acids, protein:nucleic acid complexes, carbohydrates, lipid complexes, organic and inorganic molecules, including natural and synthetic versions of any such molecules. The target or target molecules may include a single protein or other biomolecule or multiple molecules (e.g., in a multi-molecular complex). As described herein, target molecules can be tagged to facilitate detection and immobilization of bivalent binding agents of interest. Such tags include, e.g., a His-tag, FLAG-tag, V5-tag, HA-tag, or c-myc-tag. In particular embodiments, the target molecule is biotinylated.

To facilitate the practice of the present method, this invention also provides a kit for generating high affinity bivalent binding agents. The kit includes at least one set of primers for amplifying nucleic acids encoding a population of binding agents that bind to a target molecule. Such primers can be readily designed by the skilled artisan based upon the known nucleic acid sequences encoding scaffolds (i.e., non-variable regions) of antibody fragments, single-domain antibodies, FHA domains, monobodies, minibodies, scFv, AFFIBODY molecules, affilins, anticalins, DARPins and nanofitins. For example, PCR amplification of the sequence encoding the 10^{th} subunit of human fibronectin type III repeat (FN3) has been described by Karatan, et al. ((2004) Chem. Biol. 11:835-44). In addition to primers, the kit also includes a single-stranded, phage-display vector harboring a first binding agent coding region and second binding agent coding region and optionally including one or more stop codons therein, wherein the first and second binding agent coding regions are in tandem and linked via a nucleic acid molecule encoding a flexible linker (See FIG. 2).

Moreover, the kit can include reagents for primer extension and ligation including a DNA polymerase without significant strand displacement activity (e.g., T7 or T4 DNA polymerase), dNTP's, a DNA ligase (e.g., T4 ligase), ATP and DTT in appropriate reaction/storage buffers. The kit may further include PCR reagents (e.g., Taq, dNTP, MgCl₂ and buffer) for PCR amplification, reagents for reverse transcription, a biotin labeling kit, strepavidin-coated magnetic beads, DNA and RNA purification columns, wash solutions, elution solutions and/or competent cells for transformation.

The following non-limiting examples are provided to further illustrate the present invention.

### Example 1

### Identification of Bivalent Binding Agent to Pak1

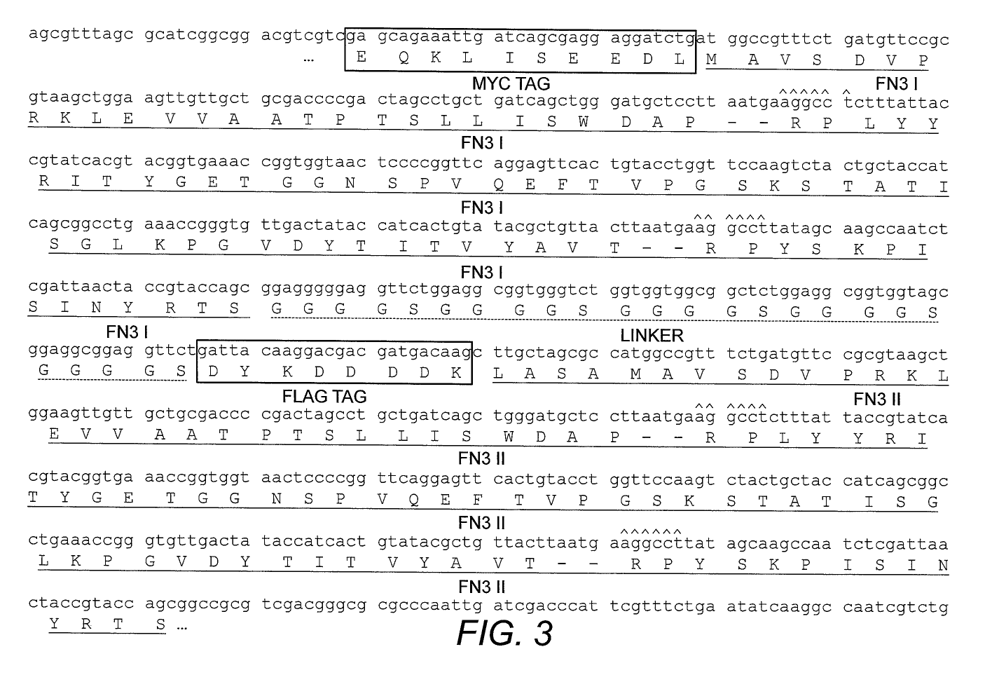

A phage-display vector was designed, which included tandem FN3 sequences containing FLAG and Myc tags for detection/capture (FIG. 2), as well as a flexible linker between the monobody sequences. In addition, to two stop codons (TAA and TGA) and StuI restriction sites were introduced into each BC and FG loop regions of the FN3 coding sequences to facilitate removal of the parent template and prevent display of the wild-type FN3 domain. More specifically, FN3 sequences were amplified with forward (5′-atg gcc gtt tct gat gtt ccg cgt a-3′; SEQ ID NO:10) and reverse (5′-gcc get ggt acg gta gtt aat cga g-3′; SEQ ID NO:11) FN3 primers and cloned in tandem into the pAP-III₆ phagemid vector (Pershad, et al. (2011) Anal. Biochem. 412:210-16; Haidaris, et al. (2001) J. Immunol. Methods 257:185-202) using SLiCE (Seamless Ligation Cloning Extract) methodology (Zhang, et al. (2014) Methods Mol. Biol. 1116:235-44). The recombinant vector was transformed into the E. coli strain TG1 (Lucigen, Madison, Wis.), which encodes wild-type versions of dUTPase and uracil-N glyosylase, to propagate the newly synthesized vector. Eight clones were selected for sequence analysis and six of the eight were identified as containing the tandem FN3 sequences (FIG. 3). Recombinant clones were introduced into E. coli strain CJ236 (New England BioLabs, Ipswich, Mass.), which lacks functional dUTPase and uracil-N glycosylase, to generate an uracilated, single-stranded phage-display template vector.

To demonstrate the generation of a bivalent binding agent using the method of the invention, nucleic acid sequences encoding C12 and A9 monobodies (Huang, et al. (2012) Methods 58:10-17), which bind different epitopes of Pak1 kinase, were amplified with forward and reverse FN3 primers to generate a pool of megaprimers. The megaprimers were annealed to the uracilated, single-stranded phage-display template vector and primer extended. The primer extension was performed in accordance with known methods (Huang, et al. (2012) Methods 58:10-17). Briefly, 15 pmol of megaprimer was phosphorylated for 1 hour at 37° C. The phosphorylated megaprimer was annealed to 5 pmol of uracilated ssDNA phagemid by denaturing at 95° C. for 2 minutes and slowly reducing the temperature by 1° C. per minute until 24° C. was reached. To fill in the remaining portions of the plasmid, the heteroduplexes were extended by the action of T7 DNA Polymerase (New England Biolabs) and T4 DNA Ligase (New England Biolabs). The resulting double-stranded DNA was purified on a QIAQUICK column and transformed by electroporation into 2×100 μL of the TG-1 strain of E. coli cells. Cells were allowed to recover at 150 rpm in 2 mL of warmed Recovery Media (Lucigen) for 30 minutes at 37° C. Cells were plated on two large 2×YT/Cb agar plates and incubated overnight at 30° C.

For phage display and screening, each plate was scraped into 5 mL of 2×YT media and the cells were combined and vortexed. Fifty microliters was used to inoculate 50 mL of 2×YT/Cb and grown to mid-log phase (OD₆₀₀=˜0.4). Cells were infected with M13K07 helper phage at an MOI of 10 for 30 minutes at 37° C. Cells were spun down at 4,000 rpm for 7 minutes and resuspended in 2×YT/CB/Kan media. The culture was shaken at room temperature for 18 hours. The overnight culture was spun down twice at 12,000 rpm for 10 minutes and the 50 mL supernatant was precipitated with 10 mL of 24% PEG, 3 M NaCl, and 4% PEG (final), for 20 minutes at room temperature. The precipitate was spun down at 12,000 rpm for 10 minutes to pellet the phage. The tubes were rinsed with 1 mL of PBS each, and the pellet was resuspended in 1 mL of PBS.

The phage particles were subsequently screened for expression of bivalent binding agents. MAXISORP 96-well microtiter plates were coated overnight with 1 μg/mL NEUTRAVIDIN (Thermo Scientific) at 4° C. The plates were blocked the next day with 200 μL of 1% casein blocking buffer in PBS (Thermo Scientific) for 1 hour. All washings were carried out in 300 μL of PBST, three times each, using a BIOTEK EL×405 plate washer. Plates were washed before addition of 50 μL of 1-10 nM biotinylated Pak1 in PBST for 1 hour with 500 rpm shaking. Plates were washed and the phage culture spun down for 5 minutes at 4,000 rpm and the supernatant diluted 1:1 with PBST before adding a total of 50 μL/well. This was incubated for 1 hour shaking. Plates were washed and 50 μL/well of 1:5000 anti-M13-HRP bacteriophage monoclonal antibody (GE HealthCare) in PBST was added to all wells for 1 hour with shaking. The final wash was performed 5 times and to each well was added 50 μL of the ABTS with 0.03% H₂O₂. Plates were read at absorbance 405 nm after 5, 15, and 30 minutes using a POLARSTAR OPTIMA microplate reader (BMG Labtech). The results of this analysis indicated that multiple clones in the tandem phage library displayed the c-myc tag and recognized Pak1. In particular, a 78% (37/47) recombination rate was achieved. Five of the recombinant clones were subjected to sequence analysis, which revealed that all four possible combinations of bivalent molecules were present in the phage-display library (Table 1).

TABLE 1

| | Conformation (N-terminus |
|---|---|
| Clone Designation | FN3-FN3-C-terminus) |
| 1 | C12-A9 |
| 2 | C12-A9 |
| 3 | A9-C12 |
| 4 | A9-A9 |
| 5 | C12-C12 |

In a different experiment, a pool of monobodies enriched from the affinity selection against Pak1 kinase domain was amplified by PCR to generate megaprimers. Using the method described herein, the megaprimers were used to generate a bivalent tandem display library of 2×10⁷ in size. The library was affinity-selected for two rounds and multiple clones were screened by phage ELISA to identify clones of higher affinities. Four clones with high ELISA values were sequenced and the sequences of their variable loops were listed in Table 2. Their sequences reveal that these clones share some conserved sequences, such as XKKTR (SEQ ID NO:21) and XXHVY (SEQ ID NO:22) in BC loop and ASWPW (SEQ ID NO:23) in FG loop. Their sequences also suggest that sequences in FN3-1 and FN3-2 are interchangeable (FN3-1 of B3 and D3 share similar sequences as FN3-2 of E5 and F10; the FN3-2 of B3 and D3 are similar to the FN3-1 of E5 and F10).

TABLE 2

| | FN3-1 | | | | FN3-2 | | | |
|---|---|---|---|---|---|---|---|---|
| | BC | SEQ | FG | SEQ | BC | SEQ | FG | SEQ |
| | loop | ID | loop | ID | loop | ID | loop | ID |
| Clone | 1 | NO: | 1 | NO: | 2 | NO: | 2 | NO: |
| B3 | CKKTR | 24 | ASWPW | 23 | ECHMH | 30 | DTRHY | 33 |
| D3 | WKKTR | 25 | ASWPW | 23 | SRHIY | 31 | DLYSN | 34 |
| E5 | WVHVY | 26 | WCSHL | 28 | LRKTS | 32 | ASWPW | 23 |
| F10 | RQHVY | 27 | HFTHP | 29 | WKKTR | 25 | ASWPW | 23 |

A competitive phage ELISA was used to estimate the half maximal inhibitory concentration (IC₅₀) of Clone D3. In this assay, the target (biotinylated Pak1) was immobilized on an ELISA plate. Varying concentrations of competitor (non-biotinylated Pak1) were mixed with phage particles displaying the bivalent binding agent and subsequently applied to the immobilized target. This analysis indicated that the IC₅₀ of Clone D3 was ˜1.5 nM, which is a 1000-fold improvement over the binding of the monobodies from the primary screen, which had a K_{D} (˜IC₅₀) in the single digit μM range. Therefore, each domain can bind simultaneously to the Pak1 target protein.

### Example 2

### Kit for MegaSTAR

Kit primers for amplifying nucleic acids encoding a population of binding agents that bind to a target molecule (Table 3).

TABLE 3

| | | | SEQ |
|---|---|---|---|
| | | Sequence | ID |
| Scaffold | Primer | (5′ -> 3′) | NO: |
| Monobody | Forward | atggccgtttctgatgt | 10 |
| (FN3) | | tccgcgta | |
| | Reverse | gccgctggtacggtagt | 11 |
| | | taatcgag | |
| Affilin | Forward | atggggaagatcacttt | 12 |
| (gamma-B | | ttacgaggac | |
| crystallin) | Reverse | tcaataaaaatccatca | 13 |
| | | cccgtcttaaagaacc | |
| DARPin | Forward | atgagaggatcgcatca | 14 |
| | | ccatcaccatcac* | |
| | Reverse | ttaattaagcttttgca | 15 |
| | | ggatttcagccagg | |

*Includes sequences encoding His₆ tag.

The kit also includes a single-stranded, phage-display vector containing first and second binding agent coding regions each encoding for a binding agent linked in tandem via a nucleic acid molecule encoding a flexible linker. Exemplary phage-display vector inserts (defined by the resulting fusion protein) and corresponding primers included in the kit include, but are not limited to, the combinations listed in Table 4. As the skilled artisan would appreciate, the N- and C-terminal orientations of the binding agents listed in Table 3 can be readily reversed.

TABLE 4

| Phage Display Vector Insert | |
|---|---|
| (N-terminus−>C-terminus) | Primers |
| Monobody-linker-Monobody | Monobody |
| Monobody-linker-DARPin | Monobody, DARPin |
| DARPin-Linker-DARPin | DARPin |
| Monobody-Linker-Affilin | Monobody, Affilin |
| Affilin-Linker-Affilin | Affilin |
| DARPin-Linker Affilin | DARPin, Affilin |

### Example 3

### Selection of Tandem Dimers

To facilitate the isolation of tandem dimers (i.e., bivalent binding agents that bind to different locations on the target molecule), a helix peptide can be inserted into the flexible linker located between the first and second binding agent coding regions. By way of illustration, an M13 calmodulin binding peptide (residues 577-602 of skeletal muscle myosin light chain kinase; Blumenthal, et al. (1985) Proc. Natl. Acad. Sci. USA 82:3187-91) could be used. In this respect, when both domains of the tandem dimer are bound to the target molecule, the M13 helix peptide would not be able to bind to calmodulin due to torsion on the helix. Therefore, any binding agents that fail to bind to two different locations on the target molecule can be readily removed via calmodulin-coated beads in the presence of calcium.

What is claimed is:

1. A method for generating a high affinity, bivalent binding agent to a target molecule comprising
(a) obtaining nucleic acids encoding a population of binding agents that bind to a target molecule;
(b) amplifying the nucleic acids of (a) to generate a pool of megaprimers;
(c) annealing the pool of megaprimers of (b) to a single-stranded, uracilated phage-display vector comprising a first binding agent coding region and second binding agent coding region each capable of hybridizing to the pool of megaprimers, wherein the first and second binding agent coding regions are in tandem and linked via a nucleic acid encoding a flexible linker;
(d) primer extending the annealed nucleic acids of (c) to generate a phage-display library bivalent phage clones; and
(e) screening the phage-display library to identify a bivalent phage clone that binds to the target molecule thereby generating a high affinity bivalent binding agent.

2. The method of claim 1, wherein the population of binding agents comprises a library of antibody fragments of antibodies, single-domain antibodies, Forkhead-Associated domains, monobodies, minibodies, single-chain variable fragments, AFFIBODY molecules, affilins, anticalins, designed ankyrin repeat proteins, nanofitins, linear peptides or a combination thereof.

3. A phage-display vector comprising a first binding agent coding region and second binding agent coding region, wherein the first and second binding agent coding regions are in tandem and linked via a nucleic acid encoding a flexible linker.

4. The phage-display vector of claim 3, wherein the first binding agent coding region and second binding agent coding region comprise at least one stop codon.

5. The phage-display vector of claim 3, wherein the first binding agent coding region and second binding agent coding region are the same.

6. The phage-display vector of claim 3, wherein the first binding agent coding region and second binding agent coding region are different.

7. A kit comprising the phage-display vector of claim 3 and primers for amplifying nucleic acids encoding a population of binding agents that bind to a target molecule.


SEQUENCE LISTING (34 sequences)

SEQ ID NO: 1 - protein, 94 aa, Artificial Sequence
  Synthetic polypeptide
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 2 - protein, 94 aa, Artificial Sequence
  Synthetic polypeptide
MAVSDVPRKLEVVAATPTSLLISWDAPCRKCLYYRITYGETGGNSPVQEFTVPGSKSTATISGLKPGVDYTITVYAVTRLEFISKPIISINYRI

SEQ ID NO: 3 - protein, 15 aa, Artificial Sequence
  Synthetic peptide
GGGGSGGGGSGGGGS

SEQ ID NO: 4 - protein, 25 aa, Artificial Sequence
  Synthetic peptide
GGGGSGGGGSGGGGSGGGGSGGGGS

SEQ ID NO: 5 - protein, 10 aa, Artificial Sequence
  Synthetic peptide
SAKTTPKLGG

SEQ ID NO: 6 - protein, 9 aa, Artificial Sequence
  Synthetic peptide
RADAAPTVS

SEQ ID NO: 7 - protein, 12 aa, Artificial Sequence
  Synthetic peptide
RADAAAAGGPGS

SEQ ID NO: 8 - protein, 27 aa, Artificial Sequence
  Synthetic peptide
RADAAAAGGGGSGGGGSGGGGSGGGGS

SEQ ID NO: 9 - protein, 5 aa, Artificial Sequence
  Synthetic peptide
GGGGS

SEQ ID NO: 10 - DNA, 25 bp, Artificial Sequence
  Synthetic oligonucleotide
atggccgtttctgatgttccgcgta

SEQ ID NO: 11 - DNA, 25 bp, Artificial Sequence
  Synthetic oligonucleotide
gccgctggtacggtagttaatcgag

SEQ ID NO: 12 - DNA, 27 bp, Artificial Sequence
  Synthetic oligonucleotide
atggggaagatcactttttacgaggac

SEQ ID NO: 13 - DNA, 33 bp, Artificial Sequence
  Synthetic oligonucleotide
tcaataaaaatccatcacccgtcttaaagaacc

SEQ ID NO: 14 - DNA, 30 bp, Artificial Sequence
  Synthetic oligonucleotide
atgagaggatcgcatcaccatcaccatcac

SEQ ID NO: 15 - DNA, 31 bp, Artificial Sequence
  Synthetic oligonucleotide
ttaattaagcttttgcaggatttcagccagg

SEQ ID NO: 16 - DNA, 800 bp, Artificial Sequence
  Synthetic polynucleotide
agcgtttagcgcatcggcggacgtcgtcgagcagaaattgatcagcgaggaggatctgatggccgtttctgatgttccgcgtaagctggaagttgttgctgcgaccccgactagcctgctgatcagctgggatgctccttaatgaaggcctctttattaccgtatcacgtacggtgaaaccggtggtaactccccggttcaggagttcactgtacctggttccaagtctactgctaccatcagcggcctgaaaccgggtgttgactataccatcactgtatacgctgttacttaatgaaggccttatagcaagccaatctcgattaactaccgtaccagcggagggggaggttctggaggcggtgggtctggtggtggcggctctggaggcggtggtagcggaggcggaggttctgattacaaggacgacgatgacaagcttgctagcgccatggccgtttctgatgttccgcgtaagctggaagttgttgctgcgaccccgactagcctgctgatcagctgggatgctccttaatgaaggcctctttattaccgtatcacgtacggtgaaaccggtggtaactccccggttcaggagttcactgtacctggttccaagtctactgctaccatcagcggcctgaaaccgggtgttgactataccatcactgtatacgctgttacttaatgaaggccttatagcaagccaatctcgattaactaccgtaccagcggccgcgtcgacgggcgcgcccaattgatcgacccattcgtttctgaatatcaaggccaatcgtctg

SEQ ID NO: 17 - protein, 37 aa, Artificial Sequence
  Synthetic peptide
EQKLISEEDLMAVSDVPRKLEVVAATPTSLLISWDAP

SEQ ID NO: 18 - protein, 127 aa, Artificial Sequence
  Synthetic polypeptide
RPLYYRITYGETGGNSPVQEFTVPGSKSTATISGLKPGVDYTITVYAVTRPYSKPISINYRTSGGGGSGGGGSGGGGSGGGGSGGGGSDYKDDDDKLASAMAVSDVPRKLEVVAATPTSLLISWDAP

SEQ ID NO: 19 - protein, 49 aa, Artificial Sequence
  Synthetic peptide
RPLYYRITYGETGGNSPVQEFTVPGSKSTATISGLKPGVDYTITVYAVT

SEQ ID NO: 20 - protein, 14 aa, Artificial Sequence
  Synthetic peptide
RPYSKPISINYRTS

SEQ ID NO: 21 - protein, 5 aa, Artificial Sequence
  Synthetic peptide
XKKTR

SEQ ID NO: 22 - protein, 5 aa, Artificial Sequence
  Synthetic peptide
XXHVY

SEQ ID NO: 23 - protein, 5 aa, Artificial Sequence
  Synthetic peptide
ASWPW

SEQ ID NO: 24 - protein, 5 aa, Artificial Sequence
  Synthetic peptide
CKKTR

SEQ ID NO: 25 - protein, 5 aa, Artificial Sequence
  Synthetic peptide
WKKTR

SEQ ID NO: 26 - protein, 5 aa, Artificial Sequence
  Synthetic peptide
WVHVY

SEQ ID NO: 27 - protein, 5 aa, Artificial Sequence
  Synthetic peptide
RQHVY

SEQ ID NO: 28 - protein, 5 aa, Artificial Sequence
  Synthetic peptide
WCSHL

SEQ ID NO: 29 - protein, 5 aa, Artificial Sequence
  Synthetic peptide
HFTHP

SEQ ID NO: 30 - protein, 5 aa, Artificial Sequence
  Synthetic peptide
ECHMH

SEQ ID NO: 31 - protein, 5 aa, Artificial Sequence
  Synthetic peptide
SRHIY

SEQ ID NO: 32 - protein, 5 aa, Artificial Sequence
  Synthetic peptide
LRKTS

SEQ ID NO: 33 - protein, 5 aa, Artificial Sequence
  Synthetic peptide
DTRHY

SEQ ID NO: 34 - protein, 5 aa, Artificial Sequence
  Synthetic peptide
DLYSN